(19) United States
(12) Patent Application Publication
(10) Pub. No.: US 2010/0168201 A1
(43) Pub. Date: Jul. 1, 2010

(54) Polymorphs of [R-(R*, R*) ]-2-(4-Fluorophenyl)-Beta, Delta-Dihydroxy-5-(1-Methylethyl)-3-Phenyl-4-[(Phenylamino)Carbonyl]-1H-Pyrrole-1-Heptanoic Acid Magnesium Salt (2:1)

(75) Inventors: Joy Mathew, Bangalore (IN); Chandrashekar Aswathanarayanappa, Bangalore (IN); Tom Thomas Puthiaparampil, Bangalore (IN)

Correspondence Address: Ballard Spahr LLP, SUITE 1000, 999 PEACHTREE STREET, ATLANTA, GA, 30309-3915, US

(73) Assignee: BIOCAN LIMITED, Bangalore (IN)

(21) Appl. No.: 12/085,255

(22) PCT Filed: Nov. 29, 2005
(86) PCT No.: PCT/IN2005/000383

§ 371 Date: Jul. 24, 2009

(51) Int. Cl. A61K 31/40; C07D 207/34; A61P 9/10

(52) U.S. Cl. 514/423; 548/537

(57) ABSTRACT

Crystalline and amorphous polymorphic forms of Atorvastatin magnesium and processes for their preparation are claimed.

### FIELD OF THE INVENTION

The invention pertains to crystalline and amorphous forms of atorvastatin magnesium as well as to processes for their preparation. The novel forms are useful as inhibitors of the enzyme3-hydroxy-3-methylglutaryl-coenzyme A reductase (HMG-CoA reductase).

### BACKGROUND OF THE INVENTION

The present invention relates to crystalline forms B1, B2 and amorphous form B3 of atorvastatin magnesium, i.e., [R—(R*,R*)]-2-(4-fluorophenyl)-β,δ,6-dihydroxy-5-(1-methylethyl)-3-phenyl-4-[(phenylamino)-carbonyl]-IH-pyrrole-heptanoic acid magnesium salt (2:1) (represented with FORMULA I), also known as atorvastatin magnesium, the processes for their preparation and isolation, pharmaceutical compositions which include the forms B1, B2 or B3, and a pharmaceutically acceptable carrier, and to a method of administering a therapeutic amount of the pharmaceutical composition for the treatment of hyperlipidemia and hypercholesterolemia.

The crystalline and amorphous forms have different properties due to the unique arrangement of molecules in the crystal lattice varying density of packing, and/or by varying hydrogen-bond network. Accordingly, individual crystalline and amorphous forms may be thought of as distinct solids having distinct advantageous and/or disadvantageous and/or physical properties compared to other polymorphic forms.

### SUMMARY OF THE INVENTION

The present invention provides for new polymorphic forms of atorvastatin magnesium, i.e. crystalline forms B1, B2 and amorphous form B3, characterized by X-ray powder diffraction pattern.

In another aspect, the present invention provides new processes for preparation of atorvastatin magnesium forms B1, B2 and amorphous form B3.

In another aspect, the invention provides pharmaceutical compositions and dosage forms comprising atorvastatin magnesium forms B1, B2 or B3.

A still further embodiment of the present invention is a method of treating hyperlipidemia or hypercholesteremia with a pharmaceutical composition containing a therapeutically effective amount of atorvastatin magnesium crystalline forms B1 and B2 and amorphous form B3.

### BRIEF DESCRIPTION OF THE DRAWINGS

The invention is further described by the following non-limiting examples, which refer to the accompanying FIGS. 1, 2 and 3, which are briefly described below.

FIG. 1 is a characteristic powder X-ray powder diffraction pattern of Atorvastatin magnesium crystalline form B1.

FIG. 2 is a characteristic powder diffraction pattern of Atorvastatin magnesium crystalline form B2.

FIG. 3 is a characteristic powder diffraction pattern of Atorvastatin magnesium amorphous form B3.

### DETAILED DESCRIPTION OF THE INVENTION

Surprisingly and unexpectedly, it has been invented that atorvastatin can be prepared in additional crystalline forms. Thus, the present invention provides atorvastatin magnesium (2:1) in three new polymorphic forms denominated as crystalline forms “B1”, “B2” and amorphous form “B3”.

The forms B1, B2 and B3 exhibit different physical characteristics as is evident from their X-ray powder diffraction patterns.

While the invention will be described in conjunction with these specific embodiments, it will be understood that it is not intended to limit the invention to such specific embodiments. On the contrary, it is intended to cover alternatives, modifications, and equivalents as may be included within the spirit and scope of the invention as defined by the appended claims. In the following description, numerous specific details are set forth in order to provide a thorough understanding of the present invention. The present invention may be practiced without some or all of these specific details. In other instance, well known process operations have not been described in detail, in order not to obscure the present invention.

This invention is related to crystalline forms B1, B2 and amorphous form B3 of [R—(R*,R*)]-2-(4-fluorophenyl)-β,δ-dihydroxy-5-(1-methylethyl)-3-phenyl-4-[(phenylamino)-carbonyl]-IH- pyrrole-heptanoic acid magnesium salt (2:1) having the following generic chemical structure:

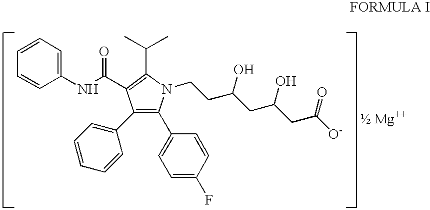

The invention is further directed to the processes for the production and isolation of forms of B1, B2 or B3, to pharmaceutical compositions which include the crystalline forms B1, B2 or amorphous form B3, and a pharmaceutically acceptable carrier, and to a method of administering a therapeutic amount of the pharmaceutical composition for the treatment of hyperlipidemia and hypercholesterolemia. The B1, B2 and B3 forms of atorvastatin magnesium are useful as inhibitors of the enzyme, 3-hydroxy-3-methylglutaryl-coenzyme A reductase, and therefore, are useful as agents for treating hyperlipidemia and hypercholesterolemia.

The B1, B2 and B3 forms are characterized by their distinctive X-ray powder diffractograms.

The present invention also provides for a method for the preparation of crystalline forms B1 and B2 and amorphous form B3 of atorvastatin magnesium (2:1). The method comprises exposing atorvastatin to different solvents and temperature conditions, which yield crystalline forms B1, B2 or amorphous form B3.

Crystalline atorvastatin magnesium form B1, B2 and amorphous atorvastatin magnesium B3 may be prepared under controlled conditions. In particular, they can be prepared/isolated by crystallization from aqueous, water-miscible, non-aqueous or non-polar solvents at a suitable temperature. Suitable solvents comprise water, acetonitrile, methanol, ethanol, acetone, ethyl acetate, chloroform, isopropyl alcohol, THF, dichloromethane, t-butanol, iso-butanol, carbon tetrachloride, 1,4-dioxan, n-butanol, di-isopropyl ether or di-ethyl ether.

In one embodiment, atorvastatin magnesium is treated with a mixture of two or more suitable solvents/anti-solvents under a suitable temperature range and the mixture can be then filtered and dried, preferably under vacuum, to obtain crystalline atorvastatin magnesium.

In another embodiment, Atorvastatin magnesium is treated with a suitable solvent or mixture of solvents under a suitable temperature range which can be then dried to obtain amorphous atorvastatin magnesium.

It will be understood that the subject to which a compound of the invention is administered need not suffer from a specific traumatic state. Indeed, the compounds of the invention may be administered prophylactically, prior to any development of symptoms. The term “therapeutic,” “therapeutically,” and permutations of these terms are used to encompass therapeutic, palliative as well as prophylactic uses. Hence, as used herein, by “treating or alleviating the symptoms” is meant reducing, preventing, and/or reversing the symptoms of the individual to which a compound of the invention has been administered, as compared to the symptoms of an individual receiving no such administration.

The term “therapeutically effective amounts used to denote treatments at dosages effective to achieve the therapeutic result sought. Furthermore, one of skill will appreciate that the therapeutically effective amount of the compound of the invention may be lowered or increased by fine tuning and/or by administering more than one compound of the invention, or by administering a compound of the invention with another compound. The invention therefore provides a method to tailor the administration/treatment to the particular exigencies specific to a given mammal. As illustrated in the following examples, therapeutically effective amounts may be easily determined for example empirically by starting at relatively low amounts and by step-wise increments with concurrent evaluation of beneficial effect.

The compounds according to the invention are optionally formulated in a pharmaceutically acceptable vehicle with any of the well known pharmaceutically acceptable carriers, including diluents and excipients (see Remington's Pharmaceutical Sciences, 18th Ed. , Gennaro, Mack Publishing Co. Easton, Pa. 1990 and Remington: The Science and Practice of Pharmacy, Lippincott, Williams & Wilkins, 1995). While the type of pharmaceutically acceptable carrier/vehicle employed in generating the compositions of the invention will vary depending upon the mode of administration of the composition to a mammal, generally pharmaceutically acceptable carriers are physiologically inert and non-toxic. Formulations of compositions according to the invention may contain more than one type of compound of the invention), as well any otherpharmacologically active ingredient useful for the treatment of the symptom/condition being treated.

The compounds of the present invention can be prepared into a pharmaceutical composition by admixing the compound with a pharmaceutically acceptable carrier, adjuvant or vehicle. The resultant pharmaceutical composition can be administered in a wide variety of dosage forms, e. g., oral, topical, parenteral or the like. It will be obvious to those skilled in the art that such dosage forms, e. g., powders, tablets, pills, capsules, aggregates, suppositories, granules and the like, or liquid forms, e.g., solutions, suspensions, or emulsions may comprise as the active component of the present invention. In solid dosage form, the atorvastatin magnesium crystalline form B1 or B2 is finely divided or mixed with one or more inactive ingredients, which can act as inactive filling materials, taste or flavor corrigenda, chemical preservatives, solubilizers, lubricants, and the like.

In liquid form, the atorvastatin magnesium crystalline form B1 or B2 is suspended, emulsified or dissolved in suitable vehicles containing various inactive components, e.g., solvents, buffers, stabilizers, colorants, flavors, and the like. The preferred unit dosages of the pharmaceutical composition of this invention typically contain from 0.5 to 100 mg of atorvastatin magnesium form B1, B2 or B3 or a mixture of forms B1, B2 and B3.

The following examples are intended to further illustrate certain preferred embodiments of the invention and are not limiting in nature. Those skilled in the art will recognize, or be able to ascertain, using no more than routine experimentation, numerous equivalents to the specific substances and procedures described herein.

### Examples

### Example 1

### Preparation of Atorvastatin Magnesium

To a solution of compound of formula II (100 g, 0.153 mol) in methanol (1.8 L), HCl (1 N, 210 mL) was added over a period of 30 minutes and stirred for 2.5 h at ambient temperature. Aqueous solution of sodium hydroxide (10%, 153 mL) was added to the reaction mixture and stirred for 2.5 h at ambient temperature. After completion of reaction (by TLC), pH of the reaction mixture was adjusted to 9.0-9.5 using 1N HCl and the mixture was filtered over celite bed. The filtrate was concentrated to about 400 mL and water (1.0 L) and methyl tert-butyl ether (MTBE, 400 mL) were added. Sufficient quantity of methanol was added to get two layers and MTBE layer was separated. Aqueous layer was further washed with MTBE (400 mL). pH of aqueous layer was adjusted to 7.5-8.0 (using 1N HCl) and washed with MTBE (2×400 mL). The aqueous layer was warmed to 40-45° C. and a solution of magnesium acetate tetra-hydrate (24.5 g, 0.114 mol) in water (570 mL) was added over a period of 1 h. After stirring the mixture at 40-45° C. for 15 minutes, it was cooled to about 30° C. over a period of 3 h. Atorvastatin magnesium was filtered and washed with a mixture of water and methanol (in the ratio 8.5:1.5).

### Example 2

### Preparation of Atorvastatin Magnesium

Compound of formula III (100 g, 0.142 mol) was suspended in a mixture of methanol (300 mL) and water (1 L) and a solution of sodium hydroxide (28.5 g) in water (90 mL) was added. The mixture was refluxed for 4 h. Reaction mixture was cooled to room temperature and washed with MTBE (400 mL). After separating layers, aqueous layer was kept under vacuum for 1 hour and the solution was allowed to stand for 2 h at room temperature. The precipitate formed was filtered. The product obtained was dissolved in a mixture of water (1 L), methanol (300 mL) and MTBE (400 mL). pH of the aqueous layer was adjusted to 7.5-8.0 with HCl (1N) and MTBE layer separated. The aqueous layer was warmed to 40-45° C. and a solution of magnesium acetate tetra-hydrate (22.9 g) in water (75 mL) was added. Reaction mixture was stirred at 40-45° C. for 1 h and cooled to ambient temperature over a period of 1 h. The product was filtered and washed with a mixture of water and methanol (in the ratio 8.5:1.5).

### Preparation of Crystalline Atorvastatin Magnesium

### Example 3

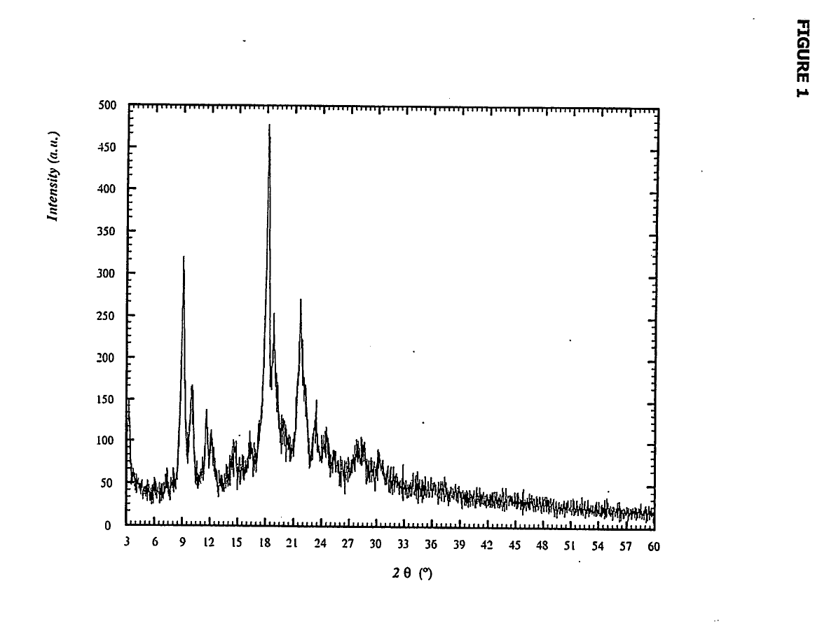

Atorvastatin Magnesium (3 g) was suspended in a mixture of acetonitrile (9 mL), water (30 mL), stirred at 35-40° C. for 20 h and filtered. The product was dried under vacuum at 40-50° C. for 12 h. Weight: 2.6 g.
XRPD: FIG. 1

### Example 4

Atorvastatin magnesium amorphous (2 g) was suspended in a mixture of methanol (9 mL), water (30 mL), heated to 35-40° C. Stirred at 35-40° C. for 20 h and filtered. The product was dried'under vacuum at 40-50° C. for 12 h. Weight: 1.7 g.
XRPD: FIG. 1

### Example 5

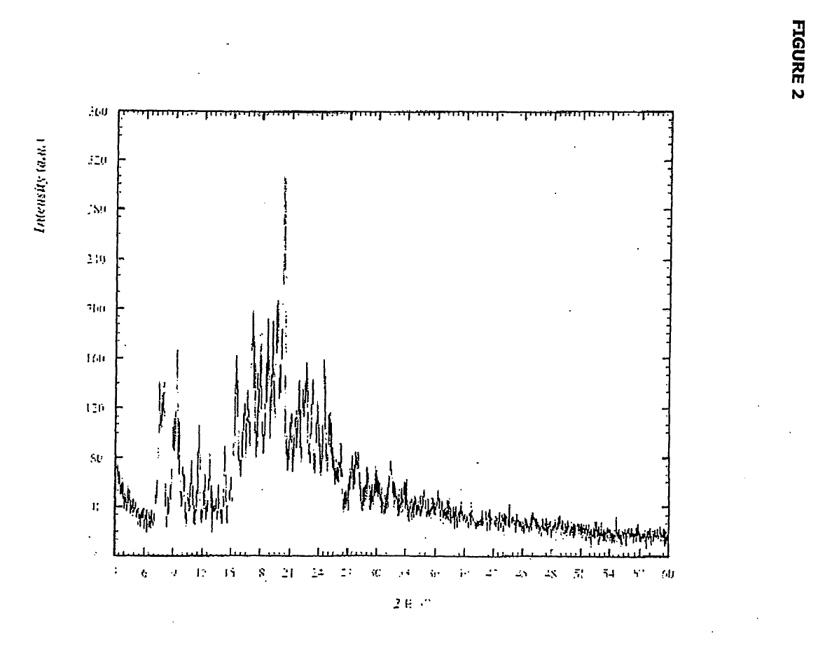

Atorvastatin magnesium (3 g) was suspended in a mixture of acetonitrile (9 mL), water (30 mL), stirred at 35-40° C. for 62 h and filtered. The product was dried under vacuum at 40-50° C. for 12 h. Weight: 2.4 g.
XRPD: FIG. 2

### Preparation of Amorphous Atorvastatin Magnesium

### Example 6

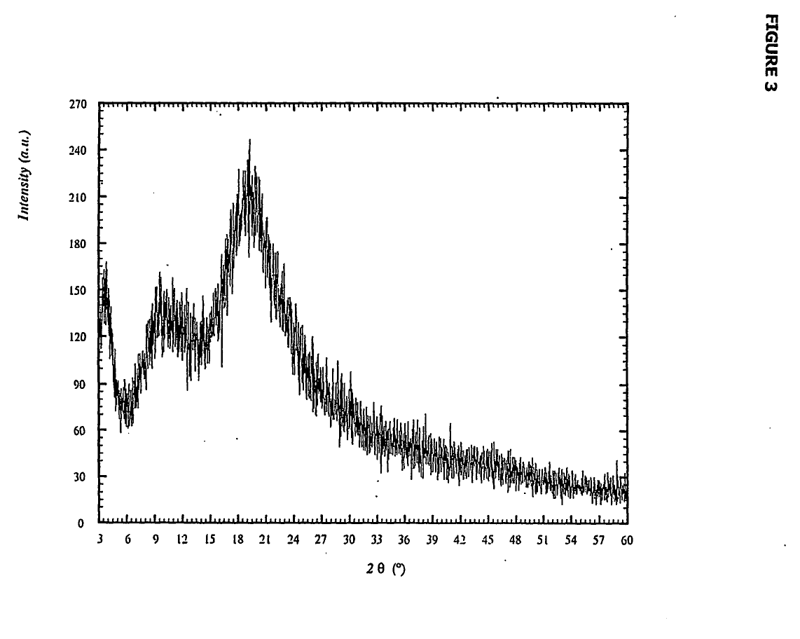

Atorvastatin magnesium (3 g) was dissolved in methanol (20 mL), frozen for 30 minutes and freeze dried. Weight: 2.8 g.
XRPD: FIG. 3

### Example 7

Atorvastatin magnesium (3 g) was dissolved in ethyl acetate (100 mL) and concentrated to 10 mL stage. Frozen for 30 minutes and freeze dried. Weight: 2.8 g.
XRPD: FIG. 3

### Example 8

Atorvastatin magnesium (3 g) was dissolved in methanol (50 mL), concentrated under vacuum at <45° C. to syrup. The syrup was transferred into a glass tray and dried at under vacuum at 40-50° C. for 12 h. Weight: 2.53 g.
XRPD: FIG. 3

### Example 9

Atorvastatin magnesium (3 g) was dissolved in a mixture of methanol (9 mL) and ethyl acetate (6 mL), concentrated under vacuum at <45° C. to syrup. The syrup was poured into a glass tray and dried at under vacuum at 40-50° C. for 12 h. Weight: 2.83 g.
XRPD: FIG. 3

### Example 10

Atorvastatin magnesium (2 g) was suspended in ethanol (40 mL), heated to 45° C., stirred for 1 h and the undissolved solids were filtered. The clear filtrate was concentrated under vacuum at <50° C. to syrup. The syrup was poured into a glass tray and dried at 25-30° C. for 2 h, then at 40-50° C. for 12 h. Weight: 1.2 g.
XRPD: FIG. 3

### Example 11

Atorvastatin magnesium (2 g) was suspended in acetone (100 mL), heated to 50° C., stirred for 1 h and the undissolved solids were filtered. The clear filtrate was concentrated under vacuum at <40° C. to syrup. The syrup was poured into a glass tray and dried at 25-30° C. for 2 h, then at 40-50° C. for 12 h. Weight: 0.9 g.
XRPD: FIG. 3

### Example 12

Atorvastatin magnesium (2 g) was suspended in THF (40 mL), heated to 45° C., stirred for 1 h and the undissolved solids were filtered. The clear filtrate was concentrated under vacuum at <50° C. to syrup. The syrup was poured into a glass tray and dried at 25-30° C. for 2 h, then at 40-50° C. for 12 h. Weight: 1.0 g.
XRPD: FIG. 3

### Example 13

Atorvastatin magnesium (2 g) was suspended in IPA (60 mL), heated to 55° C., stirred for 1 h and the undissolved solids were filtered. The clear filtrate was concentrated under vacuum at <50° C. to solid and dried at 25-30° C. for 2 h, then at 40-50° C. for 12 h. Weight: 0.9 g.
XRPD: FIG. 3

### Example 14

Atorvastatin magnesium (2 g) was suspended in acetonitrile (100 mL), heated to 45° C., stirred for 1 h and the undissolved solids were filtered. The clear filtrate was concentrated under vacuum at <50° C. to syrup. The syrup was poured into a glass tray and dried at 25-30° C. for 2 h, then at 40-50° C. for 12 h. Weight: 1.0 g.
XRPD: FIG. 3

### Example 15

Atorvastatin magnesium (2 g) was suspended chloroform (100 mL), heated to 50° C., stirred for 1 h and the undissolved solids were filtered. The clear filtrate was concentrated under vacuum at <40° C. to syrup. The syrup was poured into a glass tray and dried at 25-30° C. for 2 h, then at 40-50° C. for 12 h. Weight: 0.5 g.
XRPD: FIG. 3

### Example 16

Atorvastatin magnesium (2 g) was suspended in MDC (100 mL), heated to ˜40° C., stirred for 1 h and the undissolved solids were filtered. The clear filtrate was concentrated under vacuum at <40° C. to syrup. The syrup was poured into a glass tray and dried at 25-30° C. for 2 h, then at 40-50° C. for 12 h. Weight obtained: 1.5 g.
XRPD: FIG. 3

### Example 17

Atorvastatin magnesium (2 g) was suspended in tert-butanol (85 mL), heated to 60° C., stirred for 1 h and the undissolved solids were filtered. The clear filtrate was concentrated under vacuum at <60° C. to syrup. The syrup was poured into a glass tray and dried at 25-30° C. for 2 h, then at 40-50° C. for 12 h. Weight: 0.2 g.
XRPD: FIG. 3

### Example 18

Atorvastatin magnesium (2 g) was suspended in iso-butanol (40 mL), heated to 55° C., stirred for 1 h and the undissolved solids were filtered. The clear filtrate was concentrated under vacuum at <60° C. to syrup. The syrup was poured into a glass tray and dried at 25-30° C. for 2 h, then at 40-50° C. for 12 h. Weight: 0.2 g.
XRPD: FIG. 3

### Example 19

Atorvastatin magnesium (2 g) was suspended in carbon tetrachloride (100 mL), heated to 40° C., stirred for 1 h and the undissolved solids were filtered. The clear filtrate was concentrated under vacuum at <45° C. to syrup. The syrup was poured into a glass tray and dried at 25-30° C. for 2 h, then at 40-50° C. for 12 h. Weight: 0.3 g.
XRPD: FIG. 3

### Example 20

Atorvastatin magnesium (2 g) was suspended in 1,4-dioxan (100 mL), heated to 45° C., stirred for 1 h to dissolve, concentrated under vacuum at <50° C. to syrup. The syrup was poured into a glass tray and dried at 25-30° C. for 2 h, then at 40-50° C. for 12 h. Weight: 1.9 g.
XRPD: FIG. 3

### Example 21

Atorvastatin magnesium crude (2 g) was suspended in n-butanol (60 mL), heated to 65° C., stirred for 1 h and the undissolved solids were filtered. The clear filtrate was concentrated under vacuum at <65° C. to syrup. The syrup was poured into a glass tray and dried at 25-30° C. for 2 h, then at 40-50° C. for 12 h. Weight: 1.5 g.
XRPD: FIG. 3

### Example 22

Atorvastatin magnesium crude (2 g) was suspended in DIPE (100 mL), heated to 45° C., stirred for 1 h and the undissolved solids were filtered. The clear filtrate was concentrated under vacuum at <50° C. to syrup. The syrup was poured into a glass tray and dried at 25-30° C. for 2 h, then at 40-50° C. for 12 h. Weight: 0.3 g.
XRPD: FIG. 3

### Example 23

Atorvastatin magnesium crude (2 g) was suspended in di-ethyl ether (100 mL), heated to 40° C., stirred for 1 h and the undissolved solids were filtered. The clear filtrate was concentrated under vacuum at <40° C. to syrup. The syrup was poured into a glass tray and dried at 25-30° C. for 2 h, then at 40-50° C. for 12 h. Weight: 0.3 g.
XRPD: FIG. 3

While the salient features have been illustrated and described with respect to particular embodiments, it should be readily apparent that modifications can be made within the spirit and scope of the invention, and it is therefore not desired to limit the invention to the exact details shown and described.

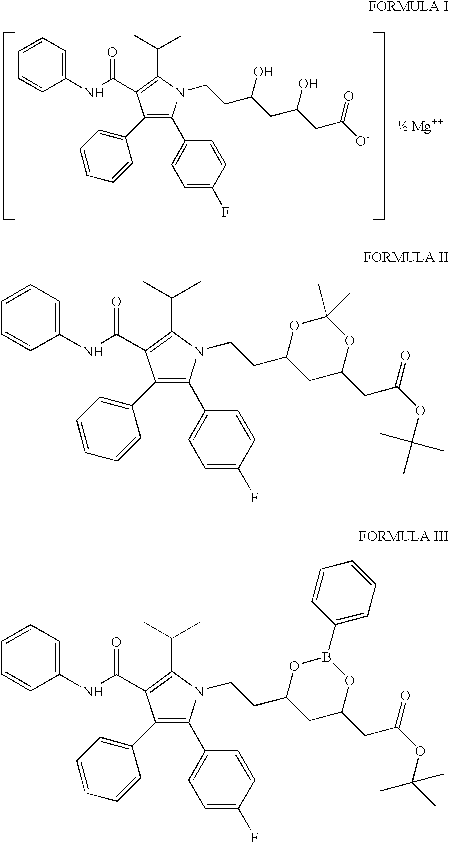

1-18. (canceled)

19. Crystalline form of Atorvastatin magnesium.

20. Amorphous form of Atorvastatin magnesium.

21. Crystalline form B1 of Atorvastatin magnesium.

22. Crystalline form B2 of Atorvastatin magnesium.

23. Amorphous form B3 of Atorvastatin magnesium.

24. The crystalline form of Atorvastatin magnesium of claim 19 having XRD pattern as shown in FIG. 1.

25. The crystalline form of Atorvastatin magnesium of claim 19 having XRD pattern as shown in FIG. 2.

26. The amorphous form of Atorvastatin magnesium of claim 20 having XRD pattern as shown in FIG. 3.

27. A process for preparation of crystalline form of Atorvastatin magnesium comprising:
a. treating Atorvastatin magnesium with one or more solvents or solvent mixtures,
b. optionally subjecting the mixture to a suitable temperature range, stirring, filtering the mixture, and
c. isolating the crystalline form of Atorvastatin magnesium.

28. A process for the preparation of amorphous form of Atorvastatin magnesium comprising:
a. treating Atorvastatin magnesium with one or more solvents or solvent mixtures, and
b. isolating the amorphous form of Atorvastatin magnesium.

29. The process of claim 27, wherein the solvent is selected from protic, aprotic, water miscible, water immiscible, polar or non-polar solvent.

30. The process of claim 28, wherein the solvent is selected from protic, aprotic, water miscible, water immiscible, polar or non-polar solvent.

31. The process of claim 29, wherein one or more solvent is selected from water, acetonitrile, methanol, ethanol, acetone, ethyl acetate, chloroform, isopropyl alcohol, THF, dichloromethane, t-butanol, iso-butanol, carbon tetrachloride, 1,4-dioxan, n-butanol, di-isopropyl ether or di-ethyl ether.

32. The process of claim 30, wherein one or more solvent is selected from water, acetonitrile, methanol, ethanol, acetone, ethyl acetate, chloroform, isopropyl alcohol, THF, dichloromethane, t-butanol, iso-butanol, carbon tetrachloride, 1,4-dioxan, n-butanol, di-isopropyl ether or di-ethyl ether.

33. The process of claim 27 to afford crystalline form B1 of Atorvastatin magnesium.

34. The process claim 28 to afford crystalline form B1 of Atorvastatin magnesium.

35. The process of claim 27 to afford crystalline form B2 of Atorvastatin magnesium.

36. The process of claim 28 to afford crystalline form B2 of Atorvastatin magnesium.

37. The process of claim 27 to afford amorphous form B3 of Atorvastatin magnesium.

38. The process of claim 28 to afford amorphous form B3 of Atorvastatin magnesium.

39. The process of claim claim 27 wherein the temperature is raised or lowered to afford the crystalline or amorphous form of atorvastatin magnesium.

40. A pharmaceutical composition comprising atorvastatin magnesium form B1, B2 or B3.

41. A method of treatment or prevention of cholesterolemia in a subject comprising administering to the subject atorvastatin magnesium form B1, B2 or B3.
